(19) United States
(12) Patent Application Publication
(10) Pub. No.: US 2006/0241078 A1
(43) Pub. Date: Oct. 26, 2006

(54) Pharmaceutical compositions comprising a contraceptive agent in solution and a teratogen

(76) Inventors: Salah U. Ahmed, New City, NY (US); Sivakumar R. Vaithiyalingam, Parsippany, NJ (US); Hemant N. Joshi, Parsippany, NJ (US)

Correspondence Address: STERNE, KESSLER, GOLDSTEIN & FOX PLLC, 1100 NEW YORK AVENUE, N.W., WASHINGTON, DC, 20005, US

(21) Appl. No.: 11/376,147

(22) Filed: Mar. 16, 2006

Related U.S. Application Data
(60) Provisional application No. 60/661,931, filed on Mar. 16, 2005.

(51) Int. Cl. A61K 31/7072; A61K 31/56; A61K 31/57; A61K 31/525; A61K 31/454; A61K 31/203

(52) U.S. Cl. 514/49; 514/170; 514/323; 514/251; 514/559

(57) ABSTRACT

The present invention relates to pharmaceutical compositions comprising an estrogen and a progestin in solution, and a teratogen suspended in the estrogen and progestin solution. The present invention also relates to methods of making and using the pharmaceutical compositions described herein.

This application claims the benefit of the filing date of U.S. Appl. 60/661,931, filed Mar. 16, 2005, which is incorporated herein in its entirety.

### BACKGROUND OF THE INVENTION

1. Field of the Invention

The present invention relates to pharmaceutical compositions comprising an estrogen and a progestin in solution, and a teratogen suspended in the estrogen and progestin solution. The present invention also relates to methods of making and using the pharmaceutical compositions described herein.

2. Background Art

Teratogens are a class of compounds that can cause malformations of an embryo or fetus. Teratogens administered to a pregnant female can be transferred to the embryo or fetus in utero, thus exposing the fetus to the harmful effects of the teratogen. Known teratogens include chemical substances, viruses and ionizing radiation. The teratogen thalidomide was previously used to control morning sickness, but was withdrawn from the market after it was discovered to cause limb and other developmental deformities. Other examples of teratogens include DES (diethylstilbestrol), a compound formerly used to prevent miscarriages before its trans-generational carcinogenic activity was known, tretinoin (retinoic acid), used for treatment of leukemia, and isotretinoin (13-cis-retinoic acid), used for the treatment of postular acne, all of which have been shown to potentially cause birth defects in developing fetuses. Due to the potential benefits that many teratogens can confer upon a patient, and the serious consequences if they are administered to a pregnant female, a practicing physician must weigh the benefits of the treatment against the possibility of pregnancy.

Numerous approaches for administering a teratogenic substance to a female capable of bearing children have been attempted, e.g. U.S. Pat. Nos. 6,702,683 and 6,428,809, and U.S. Published Application 2003/0031702 A1. U.S. Pat. Nos. 6,561,977; 6,561,976; 6,315,720 and 6,045,501 are directed to business methods for the administration of a drug to an individual without exposing a fetus to the drug. However, there continues to be a need for improved teratogen compositions which reduce the likelihood that a female can become pregnant while the teratogen is being administered.

### BRIEF SUMMARY OF THE INVENTION

The invention is directed to a pharmaceutical composition comprising:

(a) an estrogen in solution; and (b) a teratogen suspended in the estrogen solution in (a). In some embodiments, the composition further comprises a progestin. In some embodiments, the progestin is in solution.

The invention is also directed to a pharmaceutical composition comprising (a) an estrogen and a progestin in solution; (b) a teratogen suspended in the estrogen and progestin solution in (a). In some embodiments, the composition further comprises (c) a solubilizing agent. In some embodiments, the composition further comprises (d) an oil. In some embodiments, the composition further comprises a pharmaceutically acceptable excipient.

The invention is also directed to a pharmaceutical composition comprising (a) an estrogen and a progestin in solution; (b) a teratogen suspended in the estrogen and progestin solution in (a); and (c) a solubilizing agent. In some embodiments, the composition further comprises (d) an oil. In some embodiments, the composition further comprises a pharmaceutically acceptable excipient.

In some embodiments, invention is directed to a pharmaceutical composition comprising (a) an estrogen and a progestin solubilized in a solubilizing agent; and (b) a teratogen suspended in the estrogen and progestin solution in (a).

In some embodiments, the invention is directed to a pharmaceutical composition comprising: (a) a contraceptive agent; (b) a teratogen; and (c) two or more vehicles miscible to each other, wherein the contraceptive agent is in solution in at least one of the vehicles.

In some embodiments, the solubilizing agent is selected from the group consisting of an alcohol, a long-chain carboxylic acid, a polyethylene glycol ester and combinations thereof. In some embodiments, the solubilizing agent is an alcohol, and the alcohol is selected from the group consisting of ethanol, glycerol, polyethylene glycol, and combinations thereof. In some embodiments, the alcohol is ethanol. In some embodiments, the long-chain carboxylic acid is selected from the group consisting of palmitic acid, stearic acid, oleic acid, linoleic acid, linolenic acid, and combinations thereof. In some embodiments, the long-chain carboxylic acid is oleic acid. In some embodiments, the solubilizing agent is a polyethylene glycol ester.

In some embodiments, the oil is selected from the group consisting of soybean oil, vegetable oil, castor oil, mineral oil, corn oil, maize oil, hydrogenated derivatives thereof, and combinations thereof. In some embodiments, the oil is soybean oil.

In some embodiments, the composition of the present invention is suitable for oral administration. In some embodiments, the composition is in a capsule form. In some embodiments, the composition is in a liquid or semi-solid dosage form.

In some embodiments, the pharmaceutically acceptable excipient is selected from the group consisting of a stiffening agent, an antioxidant, a preservative, and combinations thereof. In some embodiments, the stiffening agent is selected from the group consisting of white wax, hydrogenated vegetable oil, polyethylene glycol, and combinations thereof. In some embodiments, the stiffening agent is white wax. In some embodiments, the antioxidant is selected from the group consisting of vitamin E, butylated hydroxyanisole, butylated hydroxytoluene, 3-dehydroshikimic acid, and combinations thereof. In some embodiments, the preservative is edetate disodium.

In some embodiments, the invention is directed to a method of making the composition of the present invention, the method comprising: (a) dissolving a contraceptive agent in a solubilizing agent to provide a contraceptive solution; (b) mixing the contraceptive solution with an oil to provide an oil-contraceptive solution; (c) adding a teratogen to an oil to provide an oil-teratogen slurry; and (d) mixing the oil-contraceptive solution of (b) and the oil-teratogen slurry of (c) to provide a teratogen-contraceptive composition.

In some embodiments, the present invention is directed to a method of contraception, the method comprising administering the composition of the present invention to a female in need thereof.

In some embodiments, the present invention is directed to a method of providing a teratogen to a female of childbearing age, the method comprising administering the composition of the present invention to the female.

### BRIEF DESCRIPTION OF THE FIGURES

FIG. 1 outlines a process flow chart for one possible method of manufacturing the pharmaceutical composition of the present invention. Part I includes combining isotretinoin with an oil, Part II includes solubilizing an estrogen and a progestin with a solubilizing agent to create an estrogen and progestin solution, then adding the estrogen and progestin solution to an oil, and Part III includes mixing the compositions of Part I and Part II.

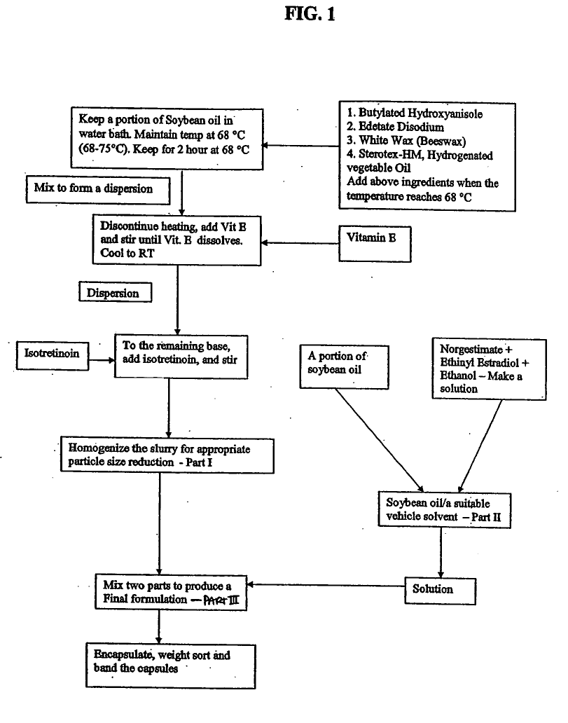

### DETAILED DESCRIPTION OF THE INVENTION

The invention is directed to a pharmaceutical composition comprising:

(a) an estrogen in solution; and (b) a teratogen suspended in the estrogen solution in (a). In some embodiments, the composition further comprises a progestin. In some embodiments, the progestin is in solution.

The invention is directed to a pharmaceutical composition comprising (a) an estrogen and a progestin in solution; and (b) a teratogen suspended in the estrogen and progestin solution in (a). In some embodiments, the composition further comprises (c) a solubilizing agent. In some embodiments, the composition further comprises (d) an oil. In some embodiments, the composition further comprises a pharmaceutically acceptable excipient.

The invention is directed to a pharmaceutical composition comprising (a) an estrogen and a progestin in solution; (b) a teratogen suspended in the estrogen and progestin solution in (a); and (c) a solubilizing agent. In some embodiments, the composition further comprises (d) an oil. In some embodiments, the composition further comprises a pharmaceutically acceptable excipient.

In some embodiments, invention is directed to a pharmaceutical composition comprising (a) an estrogen and a progestin solubilized in a solubilizing agent; and (b) a teratogen suspended in the estrogen and progestin solution in (a).

In some embodiments, the invention is directed to a pharmaceutical composition comprising: (a) a contraceptive agent; (b) a teratogen; and (c) two or more vehicles miscible to each other, wherein the contraceptive agent is in solution in at least one of the vehicles. In some embodiments, the contraceptive agent is in solution in at least two of the vehicles.

The present invention is also directed to a method of making the pharmaceutical composition described herein, the method comprising (a) dissolving a contraceptive agent in a solubilizing agent to provide a contraceptive solution, (b) mixing the contraceptive solution with an oil to provide an oil-contraceptive solution, (c) adding a teratogen to an oil to provide an oil-teratogen slurry, and (d) mixing the oil-contraceptive solution of (b) and the oil-teratogen slurry of (c) to provide a teratogen-contraceptive composition. In some embodiments, the contraceptive agent is solubilized in a solubilizing agent prior to being combined with the oil. In some embodiments, an oil/solubilizing agent mixture is used to solubilize the contraceptive agent. The term “contraceptive agent” refers to any substance that promotes the prevention of conception or impregnation. In some embodiments, a contraceptive agent is a progestin. In some embodiments, a contraceptive agent is a combination of estrogen and progestin.

Various estrogens can be used in the present invention. In some embodiments, the estrogen provides a contraceptive effect. In some embodiments, the estrogen can include, but is not limited to, estradiol, estradiol-17β, estradiol valerate, conjugated equine estrogens, piperazine estrone sulphate, estrone, estriol, estriol succinate polyestriol phosphate, ethinyl estradiol, quinestranol, mestranol, and combinations thereof. In some embodiments, the estrogen is ethinyl estradiol. In some embodiments, two or more types of estrogens can be used in a single dosage form.

Various progestins can also be used in the present invention. In some embodiments, the progestin provides a contraceptive effect. In some embodiments, the progestin can include, but is not limited to, trimegestone, norgestimate, dl-norgestrel, levonorgestrel, norethindrone (norethisterone), norethindrone acetate, ethynodiol diacetate, medroxyprogesterone acetate, cyproterone acetate, norethynodrel, gestodene, and combinations thereof. In some embodiments, the progestin is norgestimate, levonorgestrel, norethindrone (norethisterone), norethindrone acetate, or combinations thereof. In some embodiments, the progestin is norgestimate. In some embodiments, two or more types of progestins can be used in a single dosage form.

In some embodiments, the composition of the present invention can comprise both estrogen and progestin. In some embodiments, the estrogen and progestin are used for contraception. Combinations of estrogens and progestins useful for contraception are known to those in the art. In some embodiments, the combination of estrogen/progestin can include, but is not limited to, ethinyl estradiol/norgestimate, estradiol/laevo-norgestrel, estradiol-17β/laevo-norgestrel, estradiol valerate/laevo-norgestrel, conjugated equine estrogens/laevo-norgestrel, estradiol/dl-norgestrel, estradiol-17β/dl-norgestrel, estradiol valerate/dl-norgestrel, conjugated equine estrogens/dl-norgestrel, estradiol/norethindrone (norethisterone), estradiol-17β/norethindrone (norethisterone), estradiol valerate/norethindrone (norethisterone), conjugated equine estrogens/norethindrone (norethisterone), estradiol/norethindrone (norethisterone) acetate, estradiol-17β/norethindrone (norethisterone) acetate, estradiol valerate/norethindrone (norethisterone) acetate, conjugated equine estrogen/norethindrone (norethisterone) acetate, estradiol/medroxyprogesterone acetate, estradiol-17β/medroxyprogesterone acetate, estradiol valerate/medroxyprogesterone acetate, and conjugated equine estrogen/medroxyprogesterone acetate.

Various teratogens can be used in the pharmaceutical composition of the present invention. A teratogen as used herein includes any chemical or biological substance that is known to cause malformations in a developing embryo. A teratogen can be, but is not limited to, isotretinoin (Accutane®, Roche Pharmaceuticals), tretinoin (Retin-A®, Janssen-Cilag Pty Ltd.), acitretin (Soriatane®, Connetics Corporation), finasteride (Propecia®, Merck & Co.), fluorouracil (Fluoroplex®, Allergan), griseofilvin (Grifulvin®, Johnson & Johnson Pharmaceuticals), goserelin (Zoladex®, AstraZeneca), stanozol (Winstrol®, Upjohn), tazarotene (Tazorac®, Allergan), methotrexate, and thalidomide (Thalomid®, Celgene). In some embodiments, the teratogen is selected from the group consisting of isotretinoin, tretinoin, acitretin, fluorouracil, methotrexate, and thalidomide. In some embodiments, the teratogen is isotretinoin.

In some embodiments, the estrogen is in solution in a solubilizing agent, forming an estrogen solution. In some embodiments, the estrogen solution is then combined with an oil. In some embodiments, the estrogen solution is further in solution in an oil. In some embodiments, the solubilizing agent is miscible with the oil. In some embodiments, the estrogen is in solution, e.g., with an alcohol such as ethanol, to form an estrogen solution, and then the estrogen solution is combined with oil.

In some embodiments, the progestin is in solution in a solubilizing agent, forming a progestin solution. In some embodiments, the progestin solution is then combined with an oil. In some embodiments, the progestin solution is further in solution in an oil. In some embodiments, the solubilizing agent is miscible with the oil. In some embodiments, the progestin is in solution with, e.g., an alcohol such as ethanol, to form a progestin solution, and then the progestin solution is combined with oil.

In some embodiments, the estrogen and progestin are in solution in a solubilizing agent, forming an estrogen and progestin solution. The estrogen and progestin solution is then combined with an oil. In some embodiments, the solubilizing agent is miscible with the oil. In some embodiments, the estrogen and progestin is in solution with, e.g., an alcohol such as ethanol, to form an estrogen and progestin solution, and then the estrogen and progestin solution is combined with oil.

Estrogens and progestins in solution can be better absorbed in the gastrointestinal tract. Due to this increased absorption, a subject can be administered a reduced overall amount of estrogen and progestin compared to conventional formulations, yet still absorb similar amounts of the estrogen and progestin. Reduced amounts of estrogens and progestins can minimize possible side-effects of the estrogens and progestins in the subject.

“Solution” refers to a composition formed by mixing a desired substance with a liquid or solid, the resulting composition being a substantially homogeneous single-phase composition. The amount of estrogen or progestin in solution can vary, depending on the solubility constant for a given solubilizing agent with that estrogen or progestin. “Solubility constant” refers to the chemical equilibrium between solid and dissolved states of a compound in a given solubilizing agent at a given temperature and pressure. “Dissolving” is the act of forming a solution by mixing a solid with a liquid.

As described herein, an estrogen is “in solution” in a given solubilizing agent if its solubility constant in that solubilizing agent is greater than the solubility constant for that estrogen in soybean oil and/or hydrogenated vegetable oil. For example, if the solubility constant for ethinyl estradiol in soybean oil is X mg ethinyl estradiol/g soybean oil, then ethinyl estradiol is in solution in a given solubilizing agent if its solubility constant in that agent is greater than X. In some embodiments, an estrogen is considered “in solution” even when some of the estrogen is not solubilized, if the total amount of estrogen in the solubilizing agent exceeds the saturation point.

Likewise, a progestin is “in solution” in a given solubilizing agent if its solubility constant in that solubilizing agent is greater than the solubility constant for that progestin in soybean oil and/or hydrogenated vegetable oil. For example, if the solubility constant for norgestimate in soybean oil is X mg norgestimate/g soybean oil, then norgestimate is in solution in a given solubilizing agent if its solubility constant in that agent is greater than X. In some embodiments, an progestin is considered “in solution” even when some of the progestin is not solubilized, if the total amount of progestin in the solubilizing agent exceeds the saturation point.

In some embodiments, when a solubility agent is added to an oil, the combined solubility agent and oil can have a higher solubility constant for a given substance than oil by itself. For example, when ethanol is added to an oil, the solubility constant for a progestin in the oil/ethanol combination is greater than the solubility constant for progestin in oil. Since the solubility constant for progestin in the ethanol/oil combination is greater that the solubility constant in oil, then the progestin would be considered “in solution” in the ethanol/oil combination.

As described herein, the term “solubilized in a solubilizing agent” when referring to estrogens or progestins, refers to the estrogen or progestin being in solution with the given solubilizing agent.

Solubility can be determined by methods known to those in the art. For example, to determine the solubility values of the estrogen or progestin described herein, about 10 mg of estrogen (or progestin) is placed in a clear, 20 ml scintillation vial. The solubilizing agent or oil is then added drop by drop while shaking. Solubilizing agent or oil is added until the entire amount of estrogen (or progestin) is dissolved. Entrapped bubbles can be cleared, e.g., by ultrasonication. Complete solubilization can be visually confirmed under light with the naked eye.

In some embodiments, about 1% to about 100% (by weight) of the estrogen is in solution in an oil. In some embodiments, about 10% to about 100% (by weight) of the estrogen is in solution in an oil. In some embodiments, about 50% to about 100% (by weight) of the estrogen is in solution in an oil. In some embodiments, about 80% to about 100% (by weight) of the estrogen is in solution in an oil. In some embodiments, about 85% to about 100%, about 90% to about 100%, about 95% to about 100%, or about 99% to about 100% (by weight) of the estrogen is in solution an oil. In some embodiments, about 1% to about 100% (by weight) of a progestin is in solution in an oil. In some embodiments, about 10% to about 100% (by weight) of a progestin is in solution in an oil. In some embodiments, about 50% to about 100% (by weight) of a progestin is in solution in an oil. In some embodiments, about 80% to about 100% (by weight) of a progestin is in solution in an oil. In some embodiments, about 85% to about 100%, about 90% to about 100%, about 95% to about 100%, or about 99% to about 100% (by weight) of a progestin is in solution in an oil. The percentage of estrogen or progestin in solution in an oil can be determined at room temperature at about one (1) atmosphere pressure.

In some embodiments, about 70% to about 100% (by weight) of the estrogen is in solution in a solubilizing agent. In some embodiments, about 80% to about 100% (by weight) of the estrogen is in solution in a solubilizing agent. In some embodiments, about 90% to about 100% (by weight) of the estrogen is in solution in a solubilizing agent. In some embodiments, about 98% to about 100% (by weight) of the estrogen is in solution in a solubilizing agent. In some embodiments, about 99% to about 100%, about 99.5% to about 100%, or about 99.9% to about 100% (by weight) of the estrogen is in solution in a solubilizing agent. In some embodiments, about 70% to about 100% (by weight) of a progestin is in solution in a solubilizing agent. In some embodiments, about 80% to about 100% (by weight) of a progestin is in solution in a solubilizing agent. In some embodiments, about 90% to about 100% (by weight) of a progestin is in solution in a solubilizing agent. In some embodiments, about 98% to about 100% (by weight) of a progestin is in solution in a solubilizing agent. In some embodiments, about 99% to about 100%, about 99.5% to about 100%, or about 99.9% to about 100% (by weight) of a progestin is in solution in a solubilizing agent. The percentage of estrogen or progestin in solution in an oil can be determined at room temperature at about one (1) atmosphere pressure.

The concentration of estrogen in solution in a solubilizing agent can vary, as long as the concentration of estrogen in solution in the given solubilizing agent is greater than the concentration in solution in soybean and/or hydrogenated vegetable oil. In some embodiments, the concentration in solution is at least 10 mg estrogen in solution per 1 gram solubilizing agent. In some embodiments, the concentration in solution is about 10 mg to about 400 mg estrogen in solution per 1 gram solubilizing agent. In some embodiments, the concentration in solution is about 12 mg to about 300 mg, about 20 mg to about 250 mg, or about 100 to about 250 mg estrogen in solution per 1 gram of solubilizing agent.

The concentration of estrogen in solution in an oil/solubilizing agent combination can vary, as long as the concentration of estrogen in the oiI/solubilizing agent combination is greater than the concentration in solution in soybean and/or hydrogenated vegetable oil. In some embodiments, the concentration in solution is at least 10 mg estrogen in solution per 1 gram oil. In some embodiments, the concentration in solution is about 10 mg to about 400 mg estrogen in solution per 1 gram oil. In some embodiments, the concentration in solution is about 12 mg to about 300 mg, about 20 mg to about 250 mg, or about 100 to about 250 mg estrogen in solution per 1 gram of oil.

Various concentrations of progestin can also be in solution in a solubilizing agent, as long as the concentration in solution in the given solubilizing agent is greater than the concentration in solution in soybean and/or hydrogenated vegetable oil. In some embodiments, the concentration in solution is at least 2.5 mg progestin in solution per 1 gram solubilizing agent. In some embodiments, the concentration in solution is about 2.5 mg to about 100 mg progestin in solution per 1 gram solubilizing agent. In some embodiments, the concentration in solution is about 3.0 mg to about 50 mg, about 5 mg to about 30 mg, or about 10 mg to about 40 mg progestin in solution per 1 gram solubilizing agent. The concentration of estrogen or progestin in solution in a solubilizing agent is determined at room temperature at one (1) atmosphere pressure.

Various concentrations of progestin can also be in solution in an oil/solubilizing agent combination, as long as the concentration in the oil/solubilizing agent combination is greater than the concentration in solution in soybean and/or hydrogenated vegetable oil. In some embodiments, the concentration in solution is at least 2.5 mg progestin in solution per 1 gram oil. In some embodiments, the concentration in solution is about 2.5 mg to about 100 mg progestin in solution per 1 gram oil. In some embodiments, the concentration in solution is about 3.0 mg to about 50 mg, about 5 mg to about 30 mg, or about 10 mg to about 40 mg progestin in solution per 1 gram oil. The concentration of estrogen or progestin in solution in an oil is determined at room temperature at one (1) atmosphere pressure.

“Suspended” refers to the state of a substance when its particles are mixed, but not substantially dissolved, in a fluid or solid. In some embodiments, about 90% to about 100% (by weight) of the teratogen is suspended. In some embodiments, about 95% to about 100%, about 98% to about 100%, or about 99% to about 100% (by weight) of the teratogen is suspended.

In the present invention, the size of the teratogen particles suspended in oil can vary. In some embodiments, 90% of the teratogen particles are less than 400 μm in diameter. In some embodiments, 90% of the teratogen particles are less than 300 μm in diameter. In some embodiments, 90% of the teratogen particles are less than 210 μm in diameter. In some embodiments, 90% of the teratogen particles are less than 135 μm in diameter. In some embodiments, 50% of the teratogen particles are less than 200 μm in diameter. In some embodiments, 50% of the teratogen particles are less than 150 μm in diameter. In some embodiments, 50% of the teratogen particles are less than 120 μm in diameter. In some embodiments, 50% of the teratogen particles are less than 60 μm in diameter.

As used herein, the diameter of a particle is determined by measuring the diameter of a sphere having a volume equivalent to that of the particle. Particle size can be determined by various methods known to those in the art, such as, but not limited to, laser particle size detectors. For example, particle size can be determined using a Malvern Master Sizer 2000 (Worcestershire, UK).

As used herein, the term “about” refers to plus or minus 10% of the indicated number. For example, “about 90%” refers to 81% to 99%.

Various solubilizing agents can be used, as long as they are capable of dissolving the estrogen and progestin and are miscible with an oil. “Miscible” refers to the ability of the solubilizing agent and the oil to be mixed, in any ratio, without substantial separation into two phases. It would be understood by those in the art that small contaminations of either or both of the alcohol and oil, e.g., by water, that prohibit complete separation into two phrases would be within the scope of the invention. The solubilizing agents can include, but are not limited to, alcohols, long-chain carboxylic acids and polyethylene glycol esters.

In some embodiments, the alcohol is a polyol. In some embodiments, the alcohol can be, but is not limited to, ethanol, glycerol, polyethylene glycol, or combinations thereof. In some embodiments, the alcohol is ethanol.

In some embodiments, the long-chain carboxylic acid contains from 10 to 36 carbon atoms. The long-chain carboxylic acid can be saturated or unsaturated. In some embodiments, the long-chain carboxylic acid is poly unsaturated. In some embodiments, the long-chain carboxylic acid can be palmitic acid, stearic acid, oleic acid, linoleic acid, or linolenic acid. In some embodiments, the long-chain carboxylic acid is oleic acid.

In some embodiments, the solubilizing agent is a polyethylene glycol ester. A polyethylene glycol ester is a mono- or di-ester of a fatty acid or oil reacted with a polyethylene glycol. Mono or di-substituted esters of a fatty acid or oil are represented by the formula: 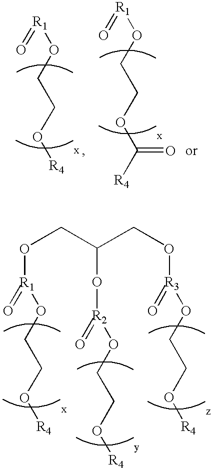
where R₁, R₂, and R₃ represent a C₈ to C₃₆ substituted or unsubstituted carbon chain, R₄ is hydrogen, or C₈ to C₃₆ substituted or unsubstituted carbon chain, and x+y+z is one or greater. In some embodiments, at least one of x or y or z is 0. In some embodiments, x is 0 to about 20. In some embodiments, y is 0 to about 20. In some embodiments, z is 0 to about 20. In some embodiments, the polyethylene glycol ester is a MAPEG® ester (BASF, Mount Olive, N.J.).

For example, in some embodiments, the polyethylene glycol ester can be MAPEG 200 ML PEG ester, MAPEG 400 DO PEG ester, MAPEG 400 DOT PEG ester, MAPEG 400 ML PEG ester, MAPEG 400 MO PEG ester. MAPEG 400 MOT PEG ester, MAPEG 400ML, MAPEG 600 DO PEG ester, MAPEG 500 DOT (89) ester, MAPEG 600 DOT PEG ester, MAPEG 600 DS PEG ester, MAPEG 600 MS PEG ester, MAPEG DS PEG ester, or combinations thereof.

Various amounts of solubilizing agents can be present in the pharmaceutical composition of the present invention. In some embodiments, the solubilizing agent can be, e.g., an alcohol, and the alcohol can be about 0.01% to about 99% (by weight) of the pharmaceutical composition. In some embodiments, the alcohol can be about 0.1% to about 50% (by weight), about 0.5% to about 20% (by weight), about 1.0% to about 15%, or about 5.0% to about 10% (by weight) of the pharmaceutical composition.

In some embodiments, the solubilizing agent can be, e.g., a long-chain carboxylic acid, and the long-chain carboxylic acid can be about 0.01% to about 99% (by weight) of the pharmaceutical composition. In some embodiments, the long-chain carboxylic acid can be about 0.1% to about 50% (by weight), about 0.5% to about 20% (by weight), about 1.0% to about 15%, or about 5.0% to about 10% (by weight) of the pharmaceutical composition.

In some embodiments, the solubilizing agent can be, e.g., polyethylene glycol ester, and the polyethylene glycol ester can be about 0.01% to about 99% (by weight) of the pharmaceutical composition. In some embodiments, the polyethylene glycol ester can be about 0.1% to about 50% (by weight), about 0.5% to about 30% (by weight), about 5.0% to about 25%, or about 10.0% to about 20% (by weight) of the pharmaceutical composition.

“Oil” according to the present invention is a substance insoluble in water but soluble in an organic solvent. An oil is a fatty acid ester of glycerol (1,2,3-propanetriol). Generally, the fatty acid hydrocarbon chains each contain greater than 8 carbons. In some embodiments, each hydrocarbon chain can contain from about 12 to about 36 carbon atoms. In some embodiments, the hydrocarbon chains can contain a variety of functional groups. In some embodiments, the hydrocarbon chain can be branched. In some embodiments, the hydrocarbon chains are unsaturated or polyunsaturated. In some embodiments, the hydrocarbon chains are saturated. The degree of saturation can affect the physical state of the oil. Thus, in some embodiments, the oil is a liquid at room temperature. In some embodiments, the oil is a solid at room temperature.

In some embodiments, the oil can be, but is not limited to, vegetable, nut, and seed oils (e.g., almond oil, castor oil, coconut oil, corn oil, maize oil, cotton seed oil, jojoba oil, linseed oil, grape seed oil, rape seed oil, mustard oil, olive oil, palm and palm kernel oil, peanut oil, safflower oil, sesame oil, soybean oil, sunflower-seed oil, crambe oil, wheat germ oil, and cocoa butter), and hydrocarbon and petroleum oils (e.g., petrolatum, mineral oil, and liquid paraffin). In some embodiments, the term “oil” refers to higher fatty acids (e.g., lauric acid, myristic acid, palmitic acid, stearic acid, behenic acid, oleic acid, 12-hydroxystearic acid, undecylenic acid, tall acid, lanolin fatty acid, isostearic acid, linoleic acid, and linolenic acid) and combinations thereof. In some embodiments, the oil can be, but is not limited to, soybean oil, vegetable oil, castor oil, corn oil, maize oil, hydrogenated derivatives thereof, or combinations thereof.

Various amounts of oil can be used in the present invention. In some embodiments, the oil is about 5% to about 95% (by weight) of the pharmaceutical composition. In some embodiments, the oil is about 40% to about 80% (by weight) of the pharmaceutical composition. In some embodiments, the oil is about 50% to about 70% (by weight) of the pharmaceutical composition.

The pharmaceutical composition of the present invention can be administered in various ways. In some embodiments, the pharmaceutical composition is suitable for oral administration. “Oral” refers to delivery of the pharmaceutical composition via, or involving, the mouth. In some embodiments, the pharmaceutical composition is a syrup or a waterless elixir. In some embodiments, the pharmaceutical composition is contained in a capsule. In some embodiments, the pharmaceutical composition is a liquid or semi-solid dosage form.

The pharmaceutical composition of the present invention can comprise a pharmaceutically acceptable excipient. As used herein, “excipient” refers to a substance that is used in the formulation of the pharmaceutical composition, and, by itself, generally has little or no therapeutic value. Various pharmaceutically acceptable excipients can be used in the present invention. In some embodiments, the pharmaceutically acceptable excipient can be, but is not limited to, a stiffening agent, an antioxidant, a preservative, a coloring agent, or combinations thereof. As used herein, “pharmaceutically acceptable” refers to compounds, materials, and compositions which are, within the scope of sound medical judgment, suitable for contact with tissues of human beings and animals without excessive toxicity, irritation, allergic response, or other problem complications commensurate with a reasonable benefit/risk ratio.

A stiffening agent is any material that, when added to a liquid substance, increases the viscosity or improves the physical consistency or integrity of the liquid substance. Examples of suitable stiffening agents include, but are not limited to, hydrogenated vegetable oil, polyethylene glycol, cetyl alcohol, cetyl esters wax, microcrystalline wax, paraffin, stearyl alcohol, lauryl alcohol, miracle alcohol, cetostearyl alcohol, white wax, yellow wax, beeswax, candelilla wax, cotton wax, carnauba wax, bayberry wax, and rice-bran wax. In some embodiments, the stiffening agent is white wax.

Various antioxidants can be used. An antioxidant is a substance that inhibits oxidation or reactions promoted by oxygen or peroxides. For example, an antioxidant can inhibit deterioration by oxidative processes such as, for example, development of rancidity in oils or inactivation of an active agent in the environment of its dosage form. Examples of antioxidants include, but are not limited to, ascorbic acid and its esters, vitamin E, sodium bisulfite, sodium metabisulfite, thiourea, butylated hydroxytoluene (BHT), butylated hydroxyanisole (BHA), 3-dehydroshikimic acid (DHS), tocopherols, alkyl gallates, and chelating agents like EDTA and citric acid. In some embodiments, the antioxidant can be vitamin E, butylated hydroxyanisole, DHS or BHT. In some embodiments, the antioxidant is vitamin E.

Various preservatives can be used. A preservative is a substance that prevents or inhibits microbial growth or undesired chemical reactions. Examples of preservatives include, but are not limited to, quaternary amines, such as quaternium 15, benzalkonium chloride, cetrimide, benzethonium chloride, and imidizolidinyl urea; organic acids, such as sorbic acid, p-hydroxybenzoic acid, and benzoic acid; parabens, such as methyl paraben and propyl paraben; alcohols, such as benzyl alcohol and isopropyl alcohol; phenols, such as triclosan, chlorhexidine, and thimerosal; hydantoin derivatives; chloromethylthiazoline; methylisothiazoline; phenyoxyethol; hexetidine; chlorohexydingluconate; imidazolidinylurea; edetate disodium; and combinations thereof. In some embodiments, the preservative is edetate disodium.

Various coloring agents can be used. A “coloring agent” (or “colorant”) employed in a composition of the present invention can be one or more pharmaceutically acceptable compounds which impart a desired color to a capsule prepared from the composition. In some embodiments, the coloring agent can be added to absorb one or more wavelengths of light, and thus act as a photoprotectant for photosensitive compounds.

The present invention is directed to a pharmaceutical composition containing (a) a contraceptive agent, and (b) a teratogen, wherein the composition contains two or more vehicles miscible to each other, and wherein at least one of the vehicles is a solvent for the contraceptive agent. In some embodiments, the contraceptive agent is in solution in at least two of the vehicles. A vehicle is an inert medium in which an agent is administered, and can include solvents, carriers, or binders for the agent, including solubilizing agents and oils.

In some embodiments, the invention is a pharmaceutical composition comprising: (a) a solution comprising ethinyl estradiol and norgestimate in soybean oil and hydrogenated vegetable oil; (b) isotretinoin suspended in the solution in (a); (c) a stiffening agent, antioxidant, and a preservative; and (d) ethanol, wherein the pharmaceutical composition is in the form of a capsule. In some embodiments, the soybean oil is about 50% to about 80% (by weight) of the pharmaceutical composition. In some embodiments, the hydrogenated vegetable oil is about 2% to about 10% (by weight) of the pharmaceutical composition. In some embodiments, the isotretinoin is about 5% to about 30% (by weight) of the pharmaceutical composition.

The amount of active agent or agents in a dosage form can vary. The exact dosage amount can be selected depending upon the needs of the subject to which the active agent is being administered, as determined by a relevant person. In some embodiments, one of skill in the art can perform pharmacokinetic studies and use the results of the study to adjust the dosage amount for a subject to a suitable level. A “relevant person” as used herein, includes, for example, a physician, physician assistant, nurse practitioner, pharmacist and customer service representative. “Dosage form” refers to a single dose of one or more agents which can be administered to a patient.

In some embodiments, the dosage form contains about 1 mg to about 250 mg of isotretinoin, about 10 mg to about 100 mg of isotretinoin, or about 20 mg to about 60 mg isotretinoin. In some embodiments, the dosage form contains about 10 mg, about 20 mg, about 30 mg, or about 40 mg of isotretinoin.

In some embodiments, the dosage form contains about 1 μg to about 250 μg, about 5 μg to about 100 μg, about 5 μg to about 50 μg, about 10 μg to about 50 μg, or about 20 μg to about 50 μg ethinyl estradiol. In some embodiments, the dosage form contains about 20 μg of ethinyl estradiol. In some embodiments, the dosage form contains about 30 μg of ethinyl estradiol. In some embodiments, the dosage form contains about 35 μg of ethinyl estradiol.

In some embodiments, the dosage form contains about 1 μg to about 5 mg of norgestimate, or about 10 μg to about 1 mg of norgestimate. In some embodiments, the dosage form contains about 100 μg to about 500 μg norgestimate, or about 200 μg to about 400 μg norgestimate. In some embodiments, the dosage form contains about 200 μg, about 250 μg, about 300 μg, about 400 μg, or about 500 μg norgestimate.

In some embodiments, the dosage form contains about 1 μg to about 10 mg of trimegestone, or about 10 μg to about 5 mg of trimegestone. In some embodiments, the dosage form contains about 100 μg to about 2.5 mg trimegestone, or about 250 μg to about 2 mg trimegestone. In some embodiments, the dosage form contains about 1 mg trimegestone.

Other estrogens and progestins can vary in potency from ethinyl estradiol and norgestimate. The values given above are for ethinyl estradiol and norgestimate, and if a different estrogen or progestin is employed, an adjustment in the amount based on the relative potency can be made and are well known in the art. The correlations in potency between the various estrogens and progestins are known. See, for example, EP 0 253 607, which is hereby incorporated in its entirety by reference. Equivalent concentrations of estrogens and of progestins can be determined using either in vitro or in vivo assay methods. See, for example, Kuhl, H., Drugs 51 (2):188-215 (1996); Philibert, D., et al., Gynecol. Endocrinol. 13:316-326 (1999); and Lundeen, S., et al., J. Steroid Biochem. Molec. Biol. 78:137-143 (2001), in which the relative potentencies of various progestins are compared using both in vitro and in vivo test assays. See also, for example, Dickey, R. P., “Contraceptive Therapy,” OBG Management Supplement (October 2000), pp. 2-6. Each of these documents is hereby incorporated by reference in its entirety.

In some aspects of the invention, the estrogen and progestin of the present invention can be ethinyl estradiol and norgestimate, respectively, although other useful estrogens and progestins can be employed. The weight ratio of estrogen and progestin can be about 1:0.2 to about 1:300. In some aspects of the invention, the weight ratio of estrogen and progestin is about 1:1 to about 1:50. In other aspects of the invention, the weight ratio of estrogen and progestin is about 1:1 to about 1:10.

In some embodiments the present invention is directed to a method of contraception, the method comprising administering the composition of the present invention to a female in need thereof. In some embodiments, the present invention is directed to a method of providing a teratogen to a female of childbearing age, the method comprising administering the composition of the present invention to the female.

The invention also provides a kit comprising the pharmaceutical composition of the present invention. The kit can include one or more containers filled with one or more of the ingredients of the pharmaceutical compositions of the invention. Optionally associated with such container(s) can be a notice or printed instructions.

For example, such printed instructions can be in a form prescribed by a governmental agency regulating the manufacture, use or sale of pharmaceuticals or biological products, which notice reflects approval by the agency of the manufacture, use or sale for human administration. In some embodiments, the kit further comprises printed matter, which, e.g., provides information on the use of the pharmaceutical composition or a pre-recorded media device which, e.g., provides information on the use of the pharmaceutical composition, or a planner.

“Printed matter” can be, for example, one of a book, booklet, brochure or leaflet. The printed matter can describe the use of the pharmaceutical composition of the present invention, e.g., for contraception. Possible formats included, but are not limited to, a bullet point list, a list of frequently asked questions (FAQ) or a chart. Additionally, the information to be imparted can be illustrated in non-textual terms using pictures, graphics or other symbols.

“Pre-recorded media device” can be, for example, a visual media device, such as a videotape cassette, a DVD (digital video disk), filmstrip, 35 mm movie or any other visual media device. Alternately, pre-recorded media device can be an interactive software application, such as a CD-ROM (compact disk-read only memory) or floppy disk. Alternately, pre-recorded media device can be, for example, an audio media device, such as a record, audiocassette or audio compact disk. The information contained on the pre-recorded media device can describe the use of the pharmaceutical composition of the present invention.

A “planner” can be, for example, a weekly, a monthly, a multi-monthly, a yearly, or a multi-yearly planner. The planner can be used as a diary to monitor dosage amounts, to keep track of dosages administered, or to prepare for future events wherein taking a regularly administered pharmaceutical composition of the present invention can be difficult. Alternately, the planner can be a calendar which will provide a means to monitor when a dosage has been taken and when it has not been taken. One skilled in the art will appreciate the variety of planning tools that would be appropriate for use with the present invention.

The kit can also include a container for storing the other components of the kit. The container can be, for example, a bag, box, envelope or any other container that would be suitable for use in the present invention. In some embodiments, the container is large enough to accommodate each component and/or any administrative devices that can be necessary of the pharmaceutical composition of the present invention. However, in some cases, it can be desirable to have a smaller container which can be hidden in a patient's pocketbook, briefcase or pocket.

The present invention is also directed to a method of delivering the pharmaceutical composition of the present invention to a patient in need thereof, the method comprising (a) registering in a computer readable medium the identity of a physician permitted to prescribe the pharmaceutical composition; (b) providing the patient with counseling information concerning the risks attendant to the pharmaceutical composition; (c) obtaining informed consent from the patient to receive the pharmaceutical composition despite the attendant risks; (d) registering the patient in a computer readable medium after obtaining their informed consent; and (e) permitting the patient access to the pharmaceutical composition.

The delivery methods of the present invention involve, inter alia, registering in a computer readable storage medium physicians who are qualified to prescribe the pharmaceutical composition of the present invention. Once registered in the computer readable storage medium, the physician can be eligible to prescribe the pharmaceutical composition to a patient in need thereof. Generally speaking, in order to become registered in the computer readable storage medium, the physician can be required to comply with various aspects of, for example, providing patient education and counseling. In some embodiments, the registration of the physician in the computer readable storage medium can be achieved by providing the physician, for example, by mail, facsimile transmission, or on-line transmission, with a registration card or form, together with educational materials concerning the pharmaceutical composition of the present invention. The physician can complete the registration card or form by providing information requested therein, and the registration card or form can be returned to the manufacturer or distributor of the pharmaceutical composition of the present invention, or other authorized recipient of the registration materials, for example, by mail, facsimile transmission or on-line transmission. The physician's information in the registration card or form is then entered into the computer readable storage medium. Suitable computer readable storage media which can be employed for registration of the physicians (as well as patients, as discussed below) will be apparent to one of ordinary skill in the art, once in possession of the teaching of the present application.

In the course of examination of a patient, the physician can determine that the patient's condition can be improved by the administration of the pharmaceutical composition of the present invention. Prior to prescribing the pharmaceutical composition of the present invention, the physician can counsel the patient, for example, on the various risks and benefits associated with the pharmaceutical composition. The patient can be provided full disclosure of all the known and suspected risks associated with the pharmaceutical composition. Such counseling can be provided verbally, as well as in written form. In some embodiments, the physician can provide the patient with literature materials on the pharmaceutical composition, such as product information, educational materials, and the like.

In some embodiments, in addition to receiving counseling on the risks attendant to the pharmaceutical composition of the present invention, the methods of the invention further require the patient or legal guardian to fill out an informed consent form which is signed by the patient or legal guardian.

Upon completion of the informed consent form, the patient can be registered in a computer readable storage medium. The computer readable storage medium in which the patient is registered can be the same as, or different from, the computer readable storage medium in which the physician is registered.

The registration into one or more computer readable storage media of the physician and patient, according to the methods describe herein, provides a means to monitor and authorize access to the pharmaceutical composition of the present invention. Thus, the computer readable storage medium can serve to deny access to patients who fail to abide by the methods of the present invention. In some embodiments, access to the pharmaceutical composition of the invention is in the form of a prescription, wherein the prescribing physician is registered in a computer readable storage medium, has provided counseling to the patient concerning the attendant risks of the pharmaceutical composition, and has obtained informed consent from the patient, prior to prescribing the pharmaceutical composition to the patient in need thereof.

The present invention is also directed to methods of educating consumers about the use of a pharmaceutical composition of the invention, the method comprising distributing the pharmaceutical composition with consumer information at a point of sale. In some embodiments, the distribution will occur at a point of sale having a pharmacist or healthcare provider.

As used herein, the term “consumer information” can include, but is not limited to, an English language text, non-English language text, visual image, chart, telephone recording, website, and access to a live costumer service representative. In some embodiments of the present invention, consumer information will provide directions for use of the pharmaceutical composition of the present invention, appropriate age use, indications, contraindications, or warnings. In some embodiments, the method further comprises providing professional information to relevant persons in a position to answer consumer questions regarding the pharmaceutical composition.

As used herein, the term “professional information” includes, but is not limited to, information concerning the pharmaceutical composition of the present invention designed to enable a healthcare professional to answer costumer questions regarding the pharmaceutical composition.

All of the various embodiments or options described herein can be combined in any and all variations.

It will be readily apparent to one of ordinary skill in the relevant arts that other suitable modifications and adaptations to the compositions and methods described herein are obvious and can be made without departing from the scope of the invention or any embodiment thereof. Having now described the present invention in detail, the same will be more clearly understood by reference to the following examples, which are included herewith for purposes of illustration only and are not intended to be limiting of the invention.

### EXAMPLE 1

The solubility of estrogen and progestin was determined for various solubilizing agents. To determine solubility, about 1 mg to about 10 mg of the estrogen or progestin was taken in a clear, 20 mL scintillation vial. The solvent was added drop by drop and shook intermittently using a mechanical shaker. Solvent was added until the entire drug was completely dissolved. Any entrapped bubbles were cleared by ultrasonification. Complete solubilization was visually confirmed under light. The solubility is represented in Table 1.

TABLE 1

| | Solubility (mg drug/g solvent or oil) | | | | |
|---|---|---|---|---|---|
| | Ethinyl | | | | Norethindrone |
| Solvent/oil | estradiol | Norgestimate | Norethindrone | Levonorgestrel | Acetate |
| Myvacet 9- | 18.08 | 4.3 | 1.71 | 0.90 | 14.47 |
| 45K | | | | | |
| MAPEG 400 | 12.08 | 9.5 | 1.94 | 0.92 | 9.34 |
| DO PEG ester | | | | | |
| Crodamol | 12.03 | 3.4 | 0.82 | 0.52 | 17.94 |
| GTCC-PN | | | | | |
| Crodamol EO | 12.94 | 2.6 | 0.62 | 0.24 | 14.42 |
| Crovol A40 | 21.37 | 6.7 | 2.55 | 1.29 | 15.85 |
| Ethanol | 206 | 20.5 | 14.19 | 5.75 | 49.46 |
| Soybean oil | 5.32 | 1.9 | 0.3 | 0.16 | 6.41 |
| Supernatant* | 2.89 | 0.802 | 0.207 | 0.0788 | 3.92 |
| of soybean oil | | | | | |
| containing | | | | | |
| isotretinoin | | | | | |

*“Supernatant of soybean oil containing isotretinoin” was provided by mixing 40 mg isotretinoin particles in 1 gram of oil, then removing the isotretinoin particles by centrifugation. The supernatant is the soybean oil that remains after removal of the isotretinoin.

### EXAMPLE 2

A pharmaceutical composition of isotretinoin, norgestimate, and ethinyl estradiol was formulated. Butylated hydroxyanisole (Tenox BHA, Eastman Chemical Company), edetate disodium, white wax, and hydrogenated vegetable oil (Sterotex-HM, Abitec Corporation) were added to soybean oil (part 1; Table 2) heated to 68° C. The heated soybean oil/excipient composition was maintained at 68° C. for 2 hours, after which vitamin E was added. The composition was stirred and heated until the vitamin E was completely dissolved, then the composition was cooled to room temperature to provide an oil base. The oil base was then mixed with isotretinoin and stirred to provide an oil-isotretinoin slurry. The oil-isotretinoin slurry was milled until the desired particle size was achieved.

Separately, norgestimate and ethinyl estradiol were dissolved in ethanol to provide a contraceptive solution.

The milled oil-isotretinoin slurry was mixed with the contraceptive solution and stirred to provide an isotretinoin-contraceptive composition (Formulation 1; Table 2).

### EXAMPLE 3

A pharmaceutical composition of isotretinoin, norgestimate, and ethinyl estradiol was formulated as described in Example 2, except that norgestimate and ethinyl estradiol were dissolved in MAPEG 400 DO (PEG 400 Dioleate), MAPEG 600 DO (PEG 600 Dioleate), or Super refined oleic acid instead of ethanol, and then added to a small amount of soybean oil (part 2) (Formulations 2, 3 or 4, respectively; Table 2) to provide a contraceptive solution.

### EXAMPLE 4

Isotretinoin was formulated with a hormonal contraceptive into a capsule using various solubilizing agents as described in Table 2:

TABLE 2

| | Composition (weight in mg) | | | |
|---|---|---|---|---|
| | Formu- | Formu- | Formu- | Formu- |
| Ingredients | lation 1 | lation 2 | lation 3 | lation 4 |
| Isotretinoin | 40 | 40 | 40 | 40 |
| Norgestimate | 0.25 | 0.25 | 0.25 | 0.25 |
| Ethinyl Estradiol | 0.035 | 0.035 | 0.035 | 0.035 |
| Soybean Oil, USP (Part 1) | 147.215 | 122.215 | 122.215 | 122.215 |
| Soybean Oil, USP (Part 2) | 0 | 10 | 10 | 10 |
| Ethanol (200 proof) | 15 | 0 | 0 | 0 |
| MAPEG 400 DO | 0 | 30 | 0 | 0 |
| (PEG 400 Dioleate) | | | | |
| MAPEG 600 DO (PEG 600 | 0 | 0 | 30 | 0 |
| Dioleate) | | | | |
| Super Refined Oleic Acid | 0 | 0 | 0 | 30 |
| Hydrogenated vegetable oil, NF | 8.0 | 8.0 | 8.0 | 8.0 |
| (Sterotex HM ™) | | | | |
| White Wax, NF (Beeswax) | 8.0 | 8.0 | 8.0 | 8.0 |
| Butylated Hydroxyanisole, Food | 0.5 | 0.5 | 0.5 | 0.5 |
| Grade (Tenox BHA) | | | | |
| Edetate disodium, USP | 0.5 | 0.5 | 0.5 | 0.5 |
| Vitamin E, USP | 0.5 | 0.5 | 0.5 | 0.5 |
| (dl-alpha Tocopherol Acetate) | | | | |
| Total | 220 | 220 | 220 | 220 |

These examples illustrate possible methods of making and possible formulations of the present invention. While the invention has been particularly shown and described with reference to some embodiments thereof, it will be understood by those skilled in the art that they have been presented by way of example only, and not limitation, and various changes in form and details can be made therein without departing from the spirit and scope of the invention. Thus, the breadth and scope of the present invention should not be limited by any of the above-described exemplary embodiments, but should be defined only in accordance with the following claims and their equivalents.

All documents cited herein, including journal articles or abstracts, published or corresponding U.S. or foreign patent applications, issued or foreign patents, or any other documents, are each entirely incorporated by reference herein, including all data, tables, figures, and text presented in the cited documents.

What is claimed is:

1. A pharmaceutical composition comprising:
(a) an estrogen and a progestin in solution; and
(b) a teratogen suspended in the estrogen and progestin solution in (a).

2. A composition of claim 1, further comprising
(c) a solubilizing agent.

3. The composition of claim 2, further comprising:
(d) an oil.

4. The composition of claim 1, wherein the estrogen is ethinyl estradiol.

5. The composition of claim 1, wherein the progestin is norgestimate.

6. The composition of claim 1, wherein the teratogen is selected from the group consisting of isotretinoin, tretinoin, acitretin, fluorouracil, methotrexate, and thalidomide.

7. The composition of claim 2, wherein the solubilizing agent is selected from the group consisting of an alcohol, a long-chain carboxylic acid, a polyethylene glycol ester and combinations thereof.

8. The composition of claim 7, wherein the solubilizing agent is an alcohol, and the alcohol is selected from the group consisting of ethanol, glycerol, polyethylene glycol, and combinations thereof.

9. The composition of claim 8, wherein the alcohol is ethanol.

10. The composition of claim 7, wherein the solubilizing agent is a long-chain carboxylic acid, and the long-chain carboxylic acid is selected from the group consisting of palmitic acid, stearic acid, oleic acid, linoleic acid, linolenic acid, and combinations thereof.

11. The composition of claim 3, wherein the oil is selected from the group consisting of soybean oil, vegetable oil, castor oil, mineral oil, corn oil, maize oil, hydrogenated derivatives thereof, and combinations thereof.

12. The composition of claim 1, which is in a liquid or semi-solid dosage form.

13. The composition of claim 4, wherein the pharmaceutically acceptable excipient is selected from the group consisting of a stiffening agent, an antioxidant, a preservative, and combinations thereof.

14. The composition of claim 13, wherein the pharmaceutically acceptable excipient is a stiffening agent, and the stiffening agent is selected from the group consisting of white wax, hydrogenated vegetable oil, polyethylene glycol, and combinations thereof.

15. The composition of claim 13, wherein the pharmaceutically acceptable excipient is an antioxidant, and the antioxidant is selected from the group consisting of vitamin E, butylated hydroxyanisole, butylated hydroxytoluene, 3-dehydroshikimic acid, and combinations thereof.

16. A pharmaceutical composition comprising:
(a) a contraceptive agent;
(b) a teratogen; and
(c) two or more vehicles miscible to each other, wherein the contraceptive agent is in solution in at least one of the vehicles.

17. A method of making the composition of claim 16, the method comprising:
(a) dissolving a contraceptive agent in a solubilizing agent to provide a contraceptive solution;
(b) mixing the contraceptive solution with an oil to provide an oil-contraceptive solution;
(c) adding a teratogen to an oil to provide an oil-teratogen slurry; and
(d) mixing the oil-contraceptive solution of (b) and the oil-teratogen slurry of (c) to provide a teratogen-contraceptive composition.

18. The method of claim 17, wherein the solubilizing agent is an alcohol.

19. A method of contraception, the method comprising administering the composition of claim 1 to a female in need thereof.

20. A method of providing a teratogen to a female of childbearing age, the method comprising administering the composition of claim 1 to the female.
